(19) United States
(12) Patent Application Publication
(10) Pub. No.: US 2026/0139189 A1
(43) Pub. Date: May 21, 2026

(54) FEEDSTOCKS FOR OIL PRODUCTION AND METHODS OF USING THE SAME

(71) Applicant: Safion Renewables Inc., Louisville, CO (US)

(72) Inventors: Lauren Ann Riley Drumm, Lafayette, CO (US); Julia Rae Friedman, Boulder, CO (US); Andrew John Borchert, Arvada, CO (US); Katherine Noon, Westminster, CO (US)

(21) Appl. No.: 19/390,268

(22) Filed: Nov. 14, 2025

Related U.S. Application Data
(60) Provisional application No. 63/721,958, filed on Nov. 18, 2024.

(51) Int. Cl. C10G 3/00

(52) U.S. Cl. CPC C10G 3/50; C10G 2300/1014

(57) ABSTRACT

Disclosed herein are feedstocks and methods of culturing A. pullulans for heavy oil production. A method of producing heavy oil can include providing one or more feedstocks comprising deacetylated and mechanically refined lignocellulosic hydrolysate media and one or more nutritional carbon sources, culturing a cell population comprising A. pullulans, or a genetically modified version thereof, on the one or more feedstocks, and generating the heavy oil using the cell population.

### CROSS-REFERENCE TO RELATED APPLICATION

This application claims the benefit of priority to U.S. Provisional Patent Application Ser. No. 63/721,958 entitled “FEEDSTOCKS FOR OIL PRODUCTION AND METHODS OF USING THE SAME,” filed Nov. 18, 2024, the disclosure of which is incorporated herein in its entirety by reference.

### BACKGROUND

Biologically derived oils produced by oleaginous microorganisms are versatile bioproducts that can be used for many applications including as biosurfactants, as oil substitutes in foods, beauty products, and other consumables, and as a biofuel precursor, such as to produce sustainable aviation fuel (SAF). The FAA has set a target for at least 3 billion gallons of sustainable aviation fuel (SAF) production per year by 2030, to reduce emissions from the tough-to-decarbonize aviation industry. SAF demand from U.S. domestic airlines is growing rapidly; however, the DOE estimates only 52 million gallons of SAF was produced and imported through June 2024, and less than a third of this SAF was domestically produced.

### SUMMARY

In an example, a method of producing sustainable, microbially derived heavy oil can include: providing one or more feedstocks comprising deacetylated and mechanically refined (DMR) lignocellulosic hydrolysate media and one or more nutritional carbon sources, wherein the one or more nutritional sources comprises one or more carbohydrates; culturing a cell population comprising Aureobasidium pullulans (A. pullulans), or a genetically modified version thereof, on the one or more feedstocks; and generating the sustainable heavy oil using the cell population.

In an example, a feedstock for use in sustainable heavy oil production can include one or more deacetylated and mechanically refined (DMR) lignocellulosic hydrolysate media; and one or more nutritional carbon sources, wherein the one or more nutritional sources comprise one or more carbohydrates.

### BRIEF DESCRIPTION OF THE DRAWINGS

In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

FIG. 1A illustrates example glucose concentration data pertaining to each exemplary feedstock between 0 days and 168 hrs.

FIG. 1B shows example pH data pertaining to each exemplary feedstock between 0 hrs and 192 hrs.

FIG. 2A illustrates example glucose consumption data pertaining to each of a variety of Oil Production media with various concentrations of nutritional sources.

FIG. 2B shows example glycerol consumption data pertaining to each of a variety of Oil Production media with various concentrations of nutritional sources.

FIG. 3A illustrates example growth measurements for control strain of A. pullulans (pks::ura3) cells grown in various media with differing nutritional sources.

FIG. 3B shows example oil concentrations produced by control strain of A. pullulans (pks::ura3) cells grown in various media with differing nutritional sources.

FIG. 4A illustrates example growth on sugar sources.

FIGS. 5A to 5D illustrate example growth on glycerol.

FIGS. 6A to 6B illustrate example growth on corn mash.

FIGS. 7A to 7B illustrate example growth on CDS.

FIG. 8 illustrates example growth on DMR corn stover hydrolysate.

### DETAILED DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Provided herein are methods of producing sustainable heavy oils via fermentation, rather than from oilseed crush processes.

Biologically based oils such as palm oil, soybean oil, sunflower oil, rapeseed oil, and olive oil, and animal fats are increasingly used as feedstocks for SAF production as part of the Hydrotreated Esters and Fatty Acids (HEFA) process; however, these oils often suffer from limited availability, price volatility, and high carbon intensity. For example, use of oils derived from soy, canola, and corn within the HEFA process results in SAF with CI scores ranging between 16.3 and 42.3 g CO₂e/MJ, a relatively modest reduction from petroleum-derived jet fuel. Therefore, the continued decarbonization of the aviation industry requires production of alternative oils suitable for synthesis of biofuels such as SAF, and which can also be derived from low cost, abundant, and low carbon intensity (CI) crops, agricultural waste, and chemical streams.

One of the primary challenges with heavy oil production via fermentation is the ability to secure adequate, sustainable, and low carbon intensity (CI) supply of feedstocks. Fermentation at the bench scale most commonly occurs using pure, monosaccharide- or disaccharide-containing feedstocks such as glucose, sucrose, and fructose, which can be expensive and environmentally disadvantageous. This work expands the known feedstocks for heavy oil production via fermentation to include low carbon intensity (CI) feedstocks including corn mash, crude glycerol, lignocellulosic biomass hydrolysates, waste carbohydrates, condensed distillers solubles (CDS) and de-oiled CDS.

Lignocellulosic hydrolysate is an abundant and low-cost feedstock for fermentation, typically obtained through the pretreatment and enzymatic saccharification of lignocellulosic biomass, including plant material and agricultural residues like corn stover. Lignocellulosic hydrolysates often contain toxic inhibitors like phenolic compounds, furans, and organic acids, which are byproducts of biomass pretreatment and hydrolysis. These inhibitors can disrupt the fermentation processes, complicating the efficient conversion of lignocellulosic biomass into valuable products. Examples of lignocellulosic hydrolysate include Dilute Acid (DA) pretreatment, Deacetylated and Mechanically Refined (DMR) processing, Deacetylation and Dilute Acid (DDA) processing, and Reductive Catalytic Fractionation (RCF) of corn stover, poplar, or switchgrass.

CDS is a byproduct of the corn ethanol industry and most commonly used as an animal feed supplement. However, owing to its nutritional profile, CDS can also act as a suitable microbial nutrient for the fermentative production of heavy oil. Since CDS is a byproduct from the ethanol production process, the displacement lifecycle assessment methodology would result in a CI score for CDS at or near zero, making heavy oil derived from CDS a low-cost and low-CI feedstock suitable for production of SAF.

The glycerol waste stream from biodiesel production, often called crude glycerol, is a byproduct generated during the transesterification of triglycerides with methanol. It typically contains only 40-80% glycerol, with the remainder consisting of methanol, water, soaps, free fatty acids, salts, and residual catalysts such as sodium or potassium hydroxide. This impure mixture is viscous, dark brown, and has a high pH due to residual base catalysts. The CI score of crude glycerol is typically in the 5-20 kg CO₂e per kg range, making it an attractive source of carbon for heavy oil production.

A variety of technical challenges exist in the current area of heavy oil production. First, as indicated above, traditional oil feedstocks (palm, soybean, sunflower, etc.) suffer from limited availability, price volatility, and high carbon intensity. Current HEFA-process oils result in SAF with CI scores ranging between 16.3 and 42.3 g CO₂e/MJ, providing only modest reductions from petroleum-derived jet fuel. Moreover, fermentation feedstock challenges exist. That is, fermentation at bench scale commonly uses expensive and environmentally disadvantageous pure monosaccharide or disaccharide feedstocks such as glucose, sucrose, and fructose. Lignocellulosic hydrolysates contain toxic inhibitors like phenolic compounds, furans, and organic acids that are byproducts of biomass pretreatment and can disrupt fermentation processes. Additionally, most microorganisms cannot efficiently utilize multiple sugar types simultaneously, leading to incomplete substrate utilization and reduced conversion efficiency.

As mentioned above, the methods discussed herein address many of these technical challenges, by use of an expanded low CI feedstock portfolio, better microorganism selection, and adaptable laboratory techniques. The techniques herein expand known feedstocks for heavy oil production to include low carbon intensity feedstocks including corn mash, crude glycerol, lignocellulosic biomass hydrolysates, waste carbohydrates, and condensed distillers solubles (CDS).

The methods further use Aureobasidium pullulans (A. pullulans), which has unique capabilities: it can natively co-utilize glucose, xylose, and arabinose, which is uncommon in many microbes; it can produce enzymes that break down polysaccharides present in hydrolysate and CDS, like xylan; and it demonstrates native tolerance to toxic inhibitor challenges associated with lignocellulosic feedstocks.

Additionally, the methods herein include culturing A. pullulans under selective pressure conditions to develop faster-growing isolates with enhanced tolerance characteristics. This addresses the inhibitor toxicity problem through strain improvement. The methods further provide multiple extraction methods including liquid-liquid extraction, distillation, physical separation, mechanical cell disruption, supercritical fluid extraction, gravimetric separation, and flash evaporation, ensuring efficient oil recovery across different feedstock types.

The use of these low-CI feedstocks makes the resulting SAF eligible for government incentives including Renewable Identification Numbers (RINs) and 48Z tax credits, addressing the economic viability problem. The methods address the core technical challenges by reducing production costs through waste stream utilization, improving carbon intensity scores through low-CI feedstock selection, enhancing substrate utilization efficiency through multi-sugar co-metabolism, overcoming fermentation inhibition through strain selection and evolution, providing process flexibility through multiple extraction options, and enabling commercial viability through regulatory compliance.

### Certain Definitions

As used herein and in the appended claims, the singular forms “a,” “and,” and “the” include plural referents unless the context clearly dictates otherwise. Thus, for example, reference to “an agent” includes a plurality of such agents and equivalents thereof known to those skilled in the art, and so forth. When ranges are used herein for physical properties, such as molecular weight, or chemical properties, such as chemical formulae, all combinations and sub-combinations of ranges and specific embodiments therein are intended to be included. The term “about” when referring to a number or a numerical range means that the number or numerical range referred to is an approximation within experimental variability (or within statistical experimental error), and thus the number or numerical range may vary between 1% and 15% of the stated number or numerical range. The term “comprising” (and related terms such as “can comprise” or “can comprise” or “having” or “including”) is not intended to exclude that in other certain embodiments, for example, an embodiment of any composition of matter, composition, method, or process, or the like, described herein, may “consist of” or “consist essentially of” the described features.

As used herein, the term “heavy oils,” “microbial oils,” and grammatical variants thereof, refers generally to molecules within a subclass of biosurfactant glycolipids identified as polyol lipids produced by the Aureobasidium pullulans yeast-like fungus. Heavy oils are amphiphilic molecules comprising a polyol head group such as mannitol, arabitol, or glycerol, attached to three to six esterified 3,5-dihydroxydecanoic acid group tails. The 3,5-dihydroxydecanoic acid group tails may be acetylated at the hydroxyl (—OH) group. Heavy oils and microbial oils lacking the polyol head group are classified as aglycone oligo-dihydroxydecanoic acids (DDA) (formerly exophilins).

Carbon Intensity (CI), as described herein, can refer to a numerical measure of the total greenhouse gas emissions associated with producing a product (e.g. SAF). For fuels the CI score is often expressed in carbon dioxide equivalent per unit energy produced (e.g. g CO₂e/MJ), while other products may express their CI score in carbon dioxide equivalent per unit mass produced (e.g. kg CO₂e/kg). In some embodiments, CI is measured using a version of The Greenhouse gases, Regulated Emissions, and Energy use in Technologies (GREET) Model, which uses input data related to the lifecycle of various fuels and transportation systems and outputs the calculated CI, determined by assessing the total greenhouse gas emissions produced per unit of activity (e.g., as described at http://greet.anl.gov/). In some embodiments, CI is measured using the R&D GREET 2023 model.

Condensed Distillers Solubles (CDS) and de-oiled CDS can be a co-product from the dry-grind corn ethanol production process. During ethanol production, after distillation of the ethanol, the remaining water and solids, known as whole stillage, can be centrifuged to separate coarse solids from the liquid. The liquid portion, referred to as thin stillage, can then be concentrated by evaporation to produce CDS. This syrup-like substance can be rich in nutrients, including carbohydrates, proteins, fats, vitamins, and minerals and may undergo further centrifugation to separate the majority of the fats (Distillers Corn Oil, DCO) from the remaining nutrients (De-oiled CDS) De-oiled CDS can consist of 30-40% solids and these solids can consist of 5-10% carbohydrates, 10-15% glycerol, and 5-15% protein (Table 1). Both forms of CDS can be commonly used as an animal feed supplement, but due to its nutritional profile, it has potential to be a microbial nutrient as well. The displacement lifecycle assessment methodology would result in a CI score for CDS at or near zero because it is a byproduct from the ethanol production process, making heavy oil derived from CDS a low-cost and low-CI feedstock suitable for production of SAF.

TABLE 1

| CDS | Wet Basis | Dry Basis |
|---|---|---|
| Moisture (%) | 65.2 | |
| Total Solids (%) | 36.1 | |
| Total Carbohydrates Est. (%) | 8.1 | 22.4 |
| Glucan Est. (%) | 4.7 | 13.0 |
| Xylan & Xylose Est. (%) | 1.8 | 4.9 |
| Arabinan & Arabinose Est. (%) | 0.3 | 0.8 |
| Mannan & Mannose (%) | 0.4 | 1.2 |
| Galactan & Galactose (%) | 0.9 | 2.4 |
| Glycerol Est. (%) | 11.9 | 32.9 |
| Organic Acids Est. (%) | 2.2 | 6.0 |
| Succinic Acid (%) | 0.4 | 1.0 |
| Lactic Acid (%) | 1.4 | 4.0 |
| Acetic Acid (%) | 0.4 | 1.0 |
| Protein (%) | 8.0 | 22.2 |
| Acid hydrolysis fat (%) | 2.3 | 6.3 |
| Acid detergent fiber (%) | N.D. | N.D. |
| Ash (%) | 3.7 | 10.2 |
| Total digestible nutrients (%) | 30.3 | 83.9 |
| Digestible energy (Mcal/lb.) | 0.61 | 1.7 |
| Metabolizable energy (Mcal/lb.) | 0.56 | 1.5 |
| Sulfur (%) | 0.63 | 1.7 |
| Phosphorus (%) | 0.62 | 1.7 |
| Potassium (%) | 0.96 | 2.7 |
| Magnesium (%) | 0.26 | 0.7 |
| Calcium (%) | 0.02 | 0.1 |
| Sodium (%) | 0.21 | 0.6 |
| Iron (ppm) | 53 | 147.5 |
| Manganese (ppm) | 13 | 36.3 |
| Copper (ppm) | 1 | 3.9 |
| Zinc (ppm) | 44 | 122.1 |

### Methods for Producing Feedstocks

Disclosed herein is a method of producing sustainable heavy oils. In some embodiments, heavy oils can comprise non-sustainable oil replacements or analogs. In some embodiments, heavy oils can comprise fuel oils, paraffin oils, edible oils, cosmetic oils, medical oils, or any combination thereof. In some embodiments, heavy oils can comprise biologically derived oils. In some embodiments, non-sustainable edible oils can comprise olive oils, sunflower oils, canola oils, soybean oils, fish oils, coconut oils, palm oils, flaxseed oils, peanut oils, avocado oils, sesame oils, almond oils, jojoba oils, argan oils, walnut oils, safflower oils, hemp oils, cod liver oils, tea tree oils, or other non-sustainable oils.

In some embodiments, the method of producing sustainable heavy oils can comprise producing one or more feedstocks. In some embodiments, feedstocks can comprise biologically derived feedstocks. In some embodiments, feedstocks can comprise biologically derived media. In some embodiments, feedstocks can comprise lignocellulosic media. In some embodiments, media can comprise lignocellulosic hydrolysate media. In some embodiments, lignocellulosic hydrolysate media can comprise deacetylated and mechanically refined (DMR) lignocellulosic hydrolysate media. In some embodiments, lignocellulosic media can be derived from corn, wheat, barley, sorghum, rye, oats, rice, millet, triticale, spelt, quinoa, buckwheat, amaranth, fonio, teff, or any combination thereof. In some embodiments, the method of producing heavy oils can further comprise adding one or more nutritional carbon sources to the DMR media feedstocks. In some embodiments, the one or more nutritional sources can comprise one or more carbohydrates. In some embodiments, the one or more nutritional sources can comprise glucose, fructose, sucrose, lactose, maltose, galactose, starch, fiber, glycogen, cellulose, mannose, raffinose, xylose, arabinose, trehalose, glycerol, pectin, inulin, chitin, or any combination thereof.

In some embodiments, the method of producing heavy oils can further comprise facilitating growth of a cell population. In some embodiments, the cell population can comprise a bacteria cell population. In some embodiments, the cell population can comprise a eukaryotic cell population. In some embodiments, the cell population can comprise a fungal cell population. In some embodiments, the cell population can comprise an enzyme-producing cell population. In some embodiments, the cell population can comprise a cell population capable of producing amylase, cellulase, fructofuranosidase, lipase, mannase, protease, xylanase, or any combination thereof. In some embodiments, the cell population can comprise a population of A. pullulans. In some embodiments, the cell population can comprise a genetically modified cell population. In some embodiments, the cell population can comprise a genetically modified A. pullulans cell population. In some embodiments, the cell population can be engineered using DNA modification, RNA modification, or another type of genetic engineering.

In some embodiments, the DMR lignocellulosic hydrolysate media can comprise one or more of hydrolysate media, corn-derived media, carbohydrate-derived media, or any combination thereof. In some embodiments, the carbohydrate-derived media can comprise sugar beet-derived media, sugarcane-derived media or corn-derived media, or any combination thereof. In some embodiments, the carbohydrate-derived media can be derived from one or more of sugarcane, sugar beets, corn, maple trees, birch trees, agave, sorghum, pomegranates, dates, apples, grapes, barley, rice, coconut, figs, stevia, honeydew sap, yacon, or any combination thereof. In some embodiments, the DMR hydrolysate media can comprise manufacturing byproducts. In some embodiments, the manufacturing byproducts can comprise waste byproducts. In some embodiments, the waste byproducts can comprise one or more of food processing waste, brewery and distillery waste, juice production waste, dairy production waste, soft drink and beverage production waste, such as beverage waste, sugar beet sucrose extraction waste, corn milling waste, molasses wash waste, confectionary waste, food canning waste, honey processing waste, or any combination thereof.

In some embodiments, the manufacturing byproducts can comprise byproducts of fuel production, food production, beverage production, livestock feed production, cosmetics production, or any combination thereof. In some embodiments, the fuel production can comprise ethanol production. In some embodiments, the manufacturing byproducts of fuel production can comprise biofuel production byproducts, such as biofuel production waste. In some embodiments, the ethanol production byproducts can comprise corn-based ethanol production byproducts. In some embodiments, the beverage production can comprise soft drink production. In some embodiments, the corn-based byproducts can comprise corn starch, CDS, high-fructose corn syrup (HFCS), CDS, corn oil, corn gluten meal, corn fiber, corn steep liquor, corn-derived protein, or any combination thereof. In some embodiments, the corn-derived media can comprise corn mash media. In some embodiments, the corn-derived media can comprise CDS media. In some embodiments, the carbohydrate-derived media can comprise carbohydrate syrup media. In some embodiments, the carbohydrate syrup media can comprise beverage-derived syrup. In some embodiments, the beverage-derived syrup can comprise HFCS, sucrose, fructose, glucose, dextrose, maltose, or any combination thereof. In some embodiments, the beverage-derived syrup can comprise byproduct syrup. In some embodiments, the one or more carbohydrates can comprise one or more of glucose, sucrose, fructose, arabinose, xylose, HFCS, or any combination thereof.

In some embodiments, the method can further comprise extracting the produced heavy oils. In some embodiments, the method can further comprise enzymatically extracting the heavy oils. In some embodiments, the extraction can comprise liquid-liquid extraction, distillation, physical separation, mechanical cell disruption, supercritical fluid extraction, gravimetric separation, flash evaporation, or a combination thereof. In some embodiments, the extraction can comprise liquid-liquid extraction. In some embodiments, the extraction can comprise distillation. In some embodiments, the extraction can comprise physical separation. In some embodiments, the extraction can comprise mechanical cell disruption. In some embodiments, the extraction can comprise supercritical fluid extraction. In some embodiments, the extraction can comprise gravimetric separation. In some embodiments, the extraction can comprise flash evaporation. In some embodiments, the extraction can comprise liquid-liquid extraction, distillation, physical separation, and flash evaporation.

In some embodiments, the liquid-liquid extraction can comprise use of a solvent. The solvent may be an organic solvent. The solvent may be a mixture of organic solvents. In some embodiments, the solvent is chloroform, methanol, butanol, isopropanol, hexane, toluene, petroleum ether, methyl ethyl ketone (MEK), acetonitrile, ethyl acetate, or a combination thereof. In some embodiments, the solvent is chloroform. In some embodiments, the solvent is methanol. In some embodiments, the solvent is butanol. In some embodiments, the solvent is isopropanol. In some embodiments, the solvent is hexane. In some embodiments, the solvent is toluene. In some embodiments, the solvent is petroleum ether. In some embodiments, the solvent is methyl ethyl ketone (MEK). In some embodiments, the solvent is acetonitrile. In some embodiments, the solvent is ethyl acetate.

In some embodiments, the liquid-liquid extraction can comprise counter current liquid extraction.

In some embodiments, the liquid-liquid extraction is completed at any suitable temperature. In some embodiments, the liquid-liquid extraction is completed at a temperature of from about 25° C. to about 60° C. In some embodiments, the liquid-liquid extraction is completed at a temperature of from about 35° C. to about 50° C. In some embodiments, the liquid-liquid extraction is completed at a temperature of at least about 20° C., about 25° C., about 30° C., about 35° C., about 40° C., about 45° C., about 50° C., about 55° C., about 60° C., or about 65° C. In some embodiments, the liquid-liquid extraction is completed at a temperature of at most about 65° C., about 60° C., about 55° C., about 50° C., about 45° C., about 40° C., about 35° C., about 30° C., about 25° C., or about 20° C. In some embodiments, the liquid-liquid extraction is completed at a temperature of from about 20° C. to about 65° C., about 25° C. to about 65° C., about 25° C. to about 60° C., about 30° C. to about 60° C., about 20° C. to about 50° C., about 30° C. to about 50° C., or about 35° C. to about 55° C. In some embodiments, the liquid-liquid extraction is completed at a temperature of about 20° C., about 25° C., about 30° C., about 35° C., about 40° C., about 45° C., about 50° C., about 55° C., about 60° C., or about 65° C.

In some embodiments, the counter current liquid extraction can comprise counter current liquid-liquid extraction. In some embodiments, the counter current liquid-liquid extraction is continuous counter current liquid-liquid extraction. In some embodiments, the liquid-liquid extraction is completed with a dynamic flow reactor.

In some embodiments, the distillation is water stripping distillation, steam distillation, vacuum distillation, azeotropic distillation, fractional distillation, or simple distillation. In some embodiments, the distillation is water stripping distillation. In some embodiments, the distillation is steam distillation. In some embodiments, the distillation is vacuum distillation. In some embodiments, the distillation is azeotropic distillation. In some embodiments, the distillation is fractional distillation. In some embodiments, the distillation is simple distillation.

In some embodiments, the distillation can comprise use of a reboiler and a condenser.

In some embodiments, the reboiler can comprise a temperature of from about 80° C. to about 120° C. In some embodiments, the reboiler can comprise a temperature of from about 90° C. to about 110° C. In some embodiments, the reboiler can comprise a temperature of at least about 75° C., about 80° C., about 85° C., about 90° C., about 95° C., about 100° C., about 105° C., about 110° C., about 115° C., or about 120° C. In some embodiments, the reboiler can comprise a temperature of at most about 120° C., about 115° C., about 110° C., about 105° C., about 100° C., about 95° C., about 90° C., about 85° C., about 80° C., or about 75° C. In some embodiments, the reboiler can comprise a temperature of from about 75° C. to about 120° C., about 80° C. to about 120° C., about 80° C. to about 115° C., about 85° C. to about 115° C., about 75° C. to about 105° C., about 85° C. to about 105° C., or about 90° C. to about 110° C. In some embodiments, the reboiler can comprise a temperature of about 75° C., about 80° C., about 85° C., about 90° C., about 95° C., about 100° C., about 105° C., about 110° C., about 115° C., or about 120° C.

In some embodiments, the condenser can comprise a temperature of from about 50° C. to about 90° C. In some embodiments, the condenser can comprise a temperature of from about 60° C. to about 80° C. In some embodiments, the condenser can comprise a temperature of at least about 50° C., about 55° C., about 60° C., about 65° C., about 70° C., about 75° C., about 80° C., about 85° C., about 90° C., or about 95° C. In some embodiments, the condenser can comprise a temperature of at most about 95° C., about 90° C., about 85° C., about 80° C., about 75° C., about 70° C., about 65° C., about 60° C., about 55° C., or about 50° C. In some embodiments, the condenser can comprise a temperature of from about 50° C. to about 95° C., about 55° C. to about 95° C., about 55° C. to about 90° C., about 60° C. to about 90° C., about 50° C. to about 80° C., about 60° C. to about 80° C., or about 65° C. to about 85° C. In some embodiments, the condenser can comprise a temperature of about 50° C., about 55° C., about 60° C., about 65° C., about 70° C., about 75° C., about 80° C., about 85° C., about 90° C., or about 95° C.

In some embodiments, the physical separation can comprise centrifugation, decantation, or a combination thereof. In some embodiments, the physical separation can comprise centrifugation. In some embodiments, the physical separation can comprise decantation.

In some embodiments, the physical separation is completed in ambient conditions. In some embodiments, the physical separation is completed at a temperature of from about 25° C. to about 60° C. In some embodiments, the physical separation is completed at a temperature of from about 35° C. to about 50° C. In some embodiments, the physical separation is completed at a temperature of at least about 25° C., about 30° C., about 35° C., about 40° C., about 45° C., about 50° C., about 55° C., about 60° C., about 65° C., or about 70° C. In some embodiments, the physical separation is completed at a temperature of at most about 70° C., about 65° C., about 60° C., about 55° C., about 50° C., about 45° C., about 40° C., about 35° C., about 30° C., or about 25° C. In some embodiments, the physical separation is completed at a temperature of from about 25° C. to about 70° C., about 30° C. to about 70° C., about 30° C. to about 65° C., about 35° C. to about 65° C., about 25° C. to about 55° C., about 35° C. to about 55° C., or about 40° C. to about 60° C. In some embodiments, the physical separation is completed at a temperature of about 25° C., about 30° C., about 35° C., about 40° C., about 45° C., about 50° C., about 55° C., about 60° C., about 65° C., or about 70° C.

In some embodiments, the centrifugation is completed in ambient conditions. In some embodiments, the centrifugation is completed at a temperature of from about 25° C. to about 60° C. In some embodiments, the centrifugation is completed at a temperature of from about 35° C. to about 50° C. In some embodiments, the centrifugation is completed at a temperature of at least about 25° C., about 30° C., about 35° C., about 40° C., about 45° C., about 50° C., about 55° C., about 60° C., about 65° C., or about 70° C. In some embodiments, the centrifugation is completed at a temperature of at most about 70° C., about 65° C., about 60° C., about 55° C., about 50° C., about 45° C., about 40° C., about 35° C., about 30° C., or about 25° C. In some embodiments, the centrifugation is completed at a temperature of from about 25° C. to about 70° C., about 30° C. to about 70° C., about 30° C. to about 65° C., about 35° C. to about 65° C., about 25° C. to about 55° C., about 35° C. to about 55° C., or about 40° C. to about 60° C. In some embodiments, the centrifugation is completed at a temperature of about 25° C., about 30° C., about 35° C., about 40° C., about 45° C., about 50° C., about 55° C., about 60° C., about 65° C., or about 70° C.

In some embodiments, the centrifugation can comprise disk-stack centrifugation.

In some embodiments, the extraction can comprise flash evaporation. In some embodiments, the flash evaporation can comprise use of a flash drum. In some embodiments, the flash evaporation is completed at any suitable temperature. In some embodiments, the flash evaporation is completed at a temperature of from about 35° C. to about 55° C. In some embodiments, the flash evaporation is completed at a temperature of at least about 35° C., about 40° C., about 45° C., about 50° C., about 55° C., about 60° C., about 65° C., about 70° C., about 75° C., or about 80° C. In some embodiments, the flash evaporation is completed at a temperature of at most about 80° C., about 75° C., about 70° C., about 65° C., about 60° C., about 55° C., about 50° C., about 45° C., about 40° C., or about 35° C. In some embodiments, the flash evaporation is completed at a temperature of from about 35° C. to about 80° C., about 40° C. to about 80° C., about 40° C. to about 75° C., about 45° C. to about 75° C., about 35° C. to about 65° C., about 45° C. to about 65° C., or about 50° C. to about 70° C. In some embodiments, the flash evaporation is completed at a temperature of about 35° C., about 40° C., about 45° C., about 50° C., about 55° C., about 60° C., about 65° C., about 70° C., about 75° C., or about 80° C.

In some embodiments, before distillation, the method can comprise solvent-extraction, such as to purify the one or more lipids provided herein. The solvent extraction may remove or reduce cellular debris, water, or other trace components. The solvent extraction may introduce organic solvent which may be removed during the subsequent distillation.

In some embodiments, the method can comprise distillation. In some embodiments, the method can comprise reacting the one or more lipids and distilling. In some embodiments, the method can comprise reacting and distilling after the extraction of the one or more lipids (e.g., after preparation by the organism). In some embodiments, the reacting the one or more lipids and distilling produces the one or more substituted or unsubstituted (e.g., saturated or unsaturated) lactones. In some embodiments, the method can comprise reacting the lipids produced by the organism described elsewhere herein. In some embodiments, the distilling provides one or more substituted or unsubstituted (e.g., saturated or unsaturated) lactones. In some embodiments, the method can comprise reacting at least a portion of the lipids to provide one or more substituted or unsubstituted (e.g., saturated or unsaturated) lactones. In some embodiments, the method can comprise reacting at least a portion of the lipids and distilling to provide the 5-hydroxy-2-decenoic acid-6-lactone and 3,5-dihydroxydecanoic acid-6-lactone.

In some embodiments, the distillation is completed at a temperature of at least 200° C. In some embodiments, the distillation is completed at a temperature of about 180° C. to about 290° C. In some embodiments, the distillation is completed at a temperature of about 180° C. to about 230° C., about 180° C. to about 290° C., about 190° C. to about 230° C., about 190° C. to about 240° C., about 200° C. to about 230° C., about 200° C. to about 240° C., about 200° C. to about 250° C., about 200° C. to about 270° C., about 200° C. to about 290° C., about 210° C. to about 230° C., about 210° C. to about 240° C., about 210° C. to about 250° C., about 210° C. to about 270° C., about 210° C. to about 290° C., about 215° C. to about 230° C., about 215° C. to about 240° C., about 215° C. to about 250° C., about 215° C. to about 270° C., about 215° C. to about 290° C., about 220° C. to about 230° C., about 220° C. to about 240° C., about 220° C. to about 250° C., about 225° C. to about 230° C., about 225° C. to about 240° C., about 225° C. to about 250° C., about 230° C. to about 240° C., or about 230° C. to about 250° C. In some embodiments, the distillation is completed at a temperature of about 180° C., about 190° C., about 200° C., about 210° C., about 215° C., about 220° C., about 225° C., about 230° C., about 240° C., about 250° C., about 270° C., or about 290° C. In some embodiments, the distillation is completed at a temperature of from about 200° C. to about 250° C. In some embodiments, the distillation is completed at a temperature of from about 215° C. to about 235° C. In some embodiments, the distillation is completed at a temperature of at least about 180° C., about 190° C., about 200° C., about 210° C., about 215° C., about 220° C., about 225° C., about 230° C., about 240° C., about 250° C., or about 270° C. In some embodiments, the distillation is completed at a temperature of at least 200° C. In some embodiments, the distillation is completed at a temperature of at most about 225° C., about 230° C., about 240° C., about 250° C., about 270° C., or about 290° C. In some embodiments, the distillation is completed at a temperature of at least 250° C. (e.g., to thermally form the lactones). In some embodiments, the distillation is completed at a temperature of less than 200° C.

In some embodiments, the wiped film distillation is completed at a temperature of at least 200° C. In some embodiments, the wiped film distillation is completed at a temperature of about 180° C. to about 290° C. In some embodiments, the wiped film distillation is completed at a temperature of about 180° C. to about 230° C., about 180° C. to about 290° C., about 190° C. to about 230° C., about 190° C. to about 240° C., about 200° C. to about 230° C., about 200° C. to about 240° C., about 200° C. to about 250° C., about 200° C. to about 270° C., about 200° C. to about 290° C., about 210° C. to about 230° C., about 210° C. to about 240° C., about 210° C. to about 250° C., about 210° C. to about 270° C., about 210° C. to about 290° C., about 215° C. to about 230° C., about 215° C. to about 240° C., about 215° C. to about 250° C., about 215° C. to about 270° C., about 215° C. to about 290° C., about 220° C. to about 230° C., about 220° C. to about 240° C., about 220° C. to about 250° C., about 225° C. to about 230° C., about 225° C. to about 240° C., about 225° C. to about 250° C., about 230° C. to about 240° C., or about 230° C. to about 250° C. In some embodiments, the wiped film distillation is completed at a temperature of about 180° C., about 190° C., about 200° C., about 210° C., about 215° C., about 220° C., about 225° C., about 230° C., about 240° C., about 250° C., about 270° C., or about 290° C. In some embodiments, the wiped film distillation is completed at a temperature of from about 200° C. to about 250° C. In some embodiments, the wiped film distillation is completed at a temperature of from about 215° C. to about 235° C. In some embodiments, the wiped film distillation is completed at a temperature of at least about 180° C., about 190° C., about 200° C., about 210° C., about 215° C., about 220° C., about 225° C., about 230° C., about 240° C., about 250° C., or about 270° C. In some embodiments, the wiped film distillation is completed at a temperature of at least 200° C. In some embodiments, the wiped film distillation is completed at a temperature of at most about 225° C., about 230° C., about 240° C., about 250° C., about 270° C., or about 290° C. In some embodiments, the wiped film distillation is completed at a temperature of about 225° C.

In some embodiments, the distillation is completed at a temperature of less than about 200° C. In some embodiments, the distillation is completed at a temperature of at least about 100° C., about 110° C., about 120° C., about 130° C., about 140° C., about 150° C., about 160° C., about 180° C., about 190° C., or about 200° C. In some embodiments, the distillation is completed at a temperature of at most about 200° C., about 190° C., about 180° C., about 160° C., about 150° C., about 140° C., about 130° C., about 120° C., about 110° C., or about 100° C. In some embodiments, the distillation is completed at a temperature of from about 100° C. to about 200° C., about 110° C. to about 200° C., about 110° C. to about 190° C., about 120° C. to about 190° C., about 100° C. to about 160° C., about 120° C. to about 160° C., or about 130° C. to about 180° C. In some embodiments, the distillation is completed at a temperature of about 100° C., about 110° C., about 120° C., about 130° C., about 140° C., about 150° C., about 160° C., about 180° C., about 190° C., or about 200° C.

In some embodiments, the reacting and/or distillation provides a yield of the one or more substituted or unsubstituted (e.g., saturated or unsaturated) lactones of at least 30 wt. %. In some embodiments, the reacting and/or distillation provides a yield of the one or more substituted or unsubstituted (e.g., saturated or unsaturated) lactones of at least 50 wt. %. In some embodiments, the reacting and/or distillation provides a yield of the one or more substituted or unsubstituted (e.g., saturated or unsaturated) lactones of at least 70 wt. %. In some embodiments, the reacting and/or distillation provides a yield of the one or more substituted or unsubstituted (e.g., saturated or unsaturated) lactones of about 30 wt. %, 35 wt. %, 40 wt. %, 45 wt. %, 50 wt. %, 55 wt. %, 60 wt. %, 65 wt. %, 70 wt. %, 75 wt. %, 80 wt. %, 85 wt. % 90 wt. %, or 95 wt. %.

In some embodiments, the reacting and/or distillation step provides mannitol (e.g., carbohydrate) and the one or more lactones.

In some embodiments, waste streams produced from the reacting and/or distilling include water vapor.

In some embodiments, the method can comprise culturing the A. pullulans cell population under selective pressure conditions. In some embodiments, the selective pressure conditions can comprise selection for nutrient availability, temperature, pH levels, oxygen levels, light exposure, pressure, presence of toxins, presence of drugs, presence of antibiotics, salinity, humidity, presence of carbohydrates, or any combination thereof. In some embodiments, the selective pressure conditions can comprise selection for faster-growing isolates of the A. pullulans cell population. In some embodiments, the selected isolates can grow faster than non-selected isolates at a rate of 1% faster, 2% faster, 3% faster, 4% faster, 5% faster, 6% faster, 7% faster, 8% faster, 9% faster, 10% faster, 11% faster, 12% faster, 13% faster, 14% faster, 15% faster, 16% faster, 17% faster, 18% faster, 19% faster, 20% faster, 21% faster, 22% faster, 23% faster, 24% faster, 25% faster, 26% faster, 27% faster, 28% faster, 29% faster, 30% faster, 31% faster, 32% faster, 33% faster, 34% faster, 35% faster, 36% faster, 37% faster, 38% faster, 39% faster, 40% faster, 41% faster, 42% faster, 43% faster, 44% faster, 45% faster, 46% faster, 47% faster, 48% faster, 49% faster, 50% faster, 51% faster, 52% faster, 53% faster, 54% faster, 55% faster, 56% faster, 57% faster, 58% faster, 59% faster, 60% faster, 61% faster, 62% faster, 63% faster, 64% faster, 65% faster, 66% faster, 67% faster, 68% faster, 69% faster, 70% faster, 71% faster, 72% faster, 73% faster, 74% faster, 75% faster, 76% faster, 77% faster, 78% faster, 79% faster, 80% faster, 81% faster, 82% faster, 83% faster, 84% faster, 85% faster, 86% faster, 87% faster, 88% faster, 89% faster, 90% faster, 91% faster, 92% faster, 93% faster, 94% faster, 95% faster, 96% faster, 97% faster, 98% faster, 99% faster, 100% faster, 101% faster, 102% faster, 103% faster, 104% faster, 105% faster, 106% faster, 107% faster, 108% faster, 109% faster, 110% faster, 111% faster, 112% faster, 113% faster, 114% faster, 115% faster, 116% faster, 117% faster, 118% faster, 119% faster, 120% faster, 121% faster, 122% faster, 123% faster, 124% faster, 125% faster, 126% faster, 127% faster, 128% faster, 129% faster, 130% faster, 131% faster, 132% faster, 133% faster, 134% faster, 135% faster, 136% faster, 137% faster, 138% faster, 139% faster, 140% faster, 141% faster, 142% faster, 143% faster, 144% faster, 145% faster, 146% faster, 147% faster, 148% faster, 149% faster, 150% faster, 151% faster, 152% faster, 153% faster, 154% faster, 155% faster, 156% faster, 157% faster, 158% faster, 159% faster, 160% faster, 161% faster, 162% faster, 163% faster, 164% faster, 165% faster, 166% faster, 167% faster, 168% faster, 169% faster, 170% faster, 171% faster, 172% faster, 173% faster, 174% faster, 175% faster, 176% faster, 177% faster, 178% faster, 179% faster, 180% faster, 181% faster, 182% faster, 183% faster, 184% faster, 185% faster, 186% faster, 187% faster, 188% faster, 189% faster, 190% faster, 191% faster, 192% faster, 193% faster, 194% faster, 195% faster, 196% faster, 197% faster, 198% faster, 199% faster, 200% faster, or more than 200% faster.

In some embodiments, the method can further comprise evaluating the fitness of the A. pullulans cell population. In some embodiments, evaluating the fitness of the A. pullulans cell population can comprise evaluating cell count, growth rate, viability, genotype, phenotype, stress resistance, heavy oil production, carbohydrate consumption, or other characteristics of the cell population.

In some embodiments, the one or more carbohydrates can comprise between about 10% and 99% of the one or more feedstocks. In some embodiments, the one or more carbohydrates can comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the one or more feedstocks. In some embodiments, the DMR lignocellulosic hydrolysate media can comprise Oil Production media.

In some embodiments, the method can further comprise modifying the pH of the one or more feedstocks after the growth of the A. pullulans cell population. In some embodiments, modifying the pH can comprise raising pH levels. In some embodiments, modifying the pH can comprise lowering the pH levels. In some embodiments, modifying the pH can comprise modifying the pH to 1, 2, 3, 4, 5, 6, 7, 8, 9, 10, 11, 12, 13, or 14 pH.

### Feedstock for Oil Production

Disclosed herein in another embodiment is a feedstock for use in sustainable heavy oil production. In some embodiments, heavy oils can comprise fuel oils, paraffin oils, edible oils, cosmetic oils, medical oils, or any combination thereof. In some embodiments, heavy oils can comprise biologically derived oils. In some embodiments, non-sustainable edible oils can comprise olive oils, sunflower oils, canola oils, soybean oils, fish oils, coconut oils, palm oils, flaxseed oils, peanut oils, avocado oils, sesame oils, almond oils, jojoba oils, argan oils, walnut oils, safflower oils, hemp oils, cod liver oils, tea tree oils, or other non-sustainable oils. In some embodiments, feedstocks can comprise biologically based feedstocks. In some embodiments, feedstocks can comprise biologically derived media. In some embodiments, feedstocks can comprise lignocellulosic media. In some embodiments, lignocellulosic media can comprise lignocellulosic hydrolysate media. In some embodiments, lignocellulosic hydrolysate media can comprise deacetylated and mechanically refined (DMR) lignocellulosic hydrolysate media. In some embodiments, lignocellulosic media can be derived from corn, wheat, barley, sorghum, rye, oats, rice, millet, triticale, spelt, quinoa, buckwheat, amaranth, fonio, teff, or any combination thereof.

In some embodiments, the feedstock can comprise one or more deacetylated and mechanically refined (DMR) lignocellulosic hydrolysate media. In some embodiments, the feedstock can further comprise one or more nutritional carbon sources. In some embodiments, the one or more nutritional sources can comprise one or more carbohydrates. In some embodiments, the one or more nutritional sources can comprise glucose, fructose, sucrose, lactose, maltose, galactose, starch, fiber, glycogen, cellulose, mannose, raffinose, xylose, arabinose, trehalose, glycerol, pectin, inulin, chitin, or any combination thereof.

In some embodiments, the DMR lignocellulosic hydrolysate media can comprise one or more of hydrolysate media, corn-derived media, carbohydrate-derived media, or any combination thereof. In some embodiments, the one or more carbohydrates can comprise carbohydrate syrup. In some embodiments, the carbohydrate syrup can comprise one or more of sugar beet-derived syrup, sugarcane-derived syrup, or corn-derived syrup, or any combination thereof. In some embodiments, the carbohydrate syrup can comprise glucose, sucrose, fructose, arabinose, xylose, high fructose corn syrup, or any combination thereof. In some embodiments, the carbohydrate syrup can comprise a first-generation carbohydrate syrup. In some embodiments, the first-generation carbohydrate syrup can be derived at least in part from corn grain or ethanol-producing plants. In some embodiments, the first-generation carbohydrate syrup can be derived from one or more of sugarcane, sugar beets, corn, maple trees, birch trees, agave, sorghum, pomegranates, dates, apples, grapes, barley, rice, coconut, figs, stevia, honeydew sap, yacon, or any combination thereof.

In some embodiments, the A. pullulans cell population or genetically modified derivative thereof can be cultured in a variety of environmental conditions. In some embodiments, the variety of environmental conditions comprises a variety of media types. In some embodiments, the variety of environmental conditions comprises a variety of carbon nutrition sources. In some embodiments, the A. pullulans cell population or genetically modified derivative thereof can be cultured in feedstocks of the variety of environmental conditions. In some embodiments, the A. pullulans cell population or genetically modified derivative can be altered to modify one or more characteristics of cell function in the feedstock. In some embodiments, the one or more characteristics of cell function in the feedstock can comprise cell efficiency in producing heavy oil. In some embodiments, the one or more characteristics of cell function in the feedstock can comprise generating a higher yield of heavy oil. In some embodiments, the one or more characteristics of cell function in the feedstock can comprise cell efficiency in producing heavy oil in an environment of the feedstock. In some embodiments, the one or more characteristics of cell function can comprise proliferation in an environment with varying levels of toxic molecules and pH levels. In some embodiments, the carbohydrate syrup can comprise a second-generation carbohydrate syrup. In some embodiments, the second-generation carbohydrate syrup can comprise a waste carbohydrate syrup. In some embodiments, the waste carbohydrate syrup is derived at least in part from agricultural waste, food waste, beverage waste, energy production crop waste, or any combination thereof. In some embodiments, the waste carbohydrate syrup can comprise an off-specification carbohydrate syrup. In some embodiments, the beverage waste can comprise off-specification soft drink syrup.

In some embodiments, the carbohydrate syrups can be manufactured as byproducts. In some embodiments, the manufacturing byproducts can comprise waste byproducts. In some embodiments, the waste byproducts can comprise one or more of food processing waste, brewery and distillery waste, juice production waste, dairy production waste, soft drink and beverage production waste, such as sugar beet sucrose extraction waste, corn milling waste, molasses wash waste, confectionary waste, food canning waste, honey processing waste, or any combination thereof.

In some embodiments, the manufacturing byproducts can comprise byproducts of fuel production, food production, beverage production, livestock feed production, cosmetics production, or any combination thereof. In some embodiments, the fuel production can comprise ethanol production. In some embodiments, the manufacturing byproducts of fuel production can comprise biofuel production byproducts, such as biofuel production waste. In some embodiments, the ethanol production byproducts can comprise corn-based ethanol production byproducts. In some embodiments, the beverage production can comprise soft drink production. In some embodiments, the corn-based byproducts can comprise corn starch, CDS, high-fructose corn syrup (HFCS), corn oil, corn gluten meal, corn fiber, corn steep liquor, corn-derived protein, or any combination thereof. In some embodiments, the carbohydrate-derived media can comprise carbohydrate syrup media. In some embodiments, the carbohydrate syrup media can comprise beverage-derived syrup. In some embodiments, the beverage-derived syrup can comprise HFCS, sucrose, fructose, glucose, dextrose, maltose, or any combination thereof. In some embodiments, the beverage-derived syrup can comprise byproduct syrup. In some embodiments, the one or more carbohydrate syrups can comprise one or more syrups of glucose, sucrose, fructose, arabinose, xylose, HFCS, or any combination thereof.

In some embodiments, the one or more carbohydrates can comprise between about 10% and 99% of the one or more feedstocks. In some embodiments, the one or more carbohydrates can comprise about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the one or more feedstocks.

### EXAMPLES

The following examples are provided to further illustrate some embodiments of the present disclosure but are not intended to limit the scope of the disclosure; it will be understood by their exemplary nature that other procedures, methodologies, or techniques known to those skilled in the art may alternatively be used.

### Example 1: Shake Flask Fermentation

In one non-limiting embodiment, batch fermentation in shake flasks was performed. The batch fermentation was performed using corn mash-based feedstocks. Both the parent strain of A. pullulans (Δpks::ura3) and the engineered strain of A. pullulans were used, for example. Various carbon nutrition sources were used such as oil production media (OPM). Enzymes were used to convert the corn mash-derived complex carbohydrates into fermentable carbohydrates such as glucose.

### Example 2: Fed-Batch Fermentation

A baffled stir-tank bioreactor was used to culture the A. pullulans cell population with a volume of, for example, 1-10 L. In some cases, a total volume of 8 L was used.

The initial concentration of carbohydrates was, for example, between 30-150 g/L. In some examples, the initial concentration of carbohydrates was at 75 g/L.

Carbohydrate sources included, for example, DMR corn stover hydrolysate, CDS, de-oiled CDS, high fructose corn syrup, waste sucrose, glucose, fructose, xylose, and arabinose.

The carbohydrate feed began after 24-72 hours at a rate of 0.1-4 g/l/h, for example. In some examples, the carbohydrate feed was set at 0.5 g/l/h. Agitation of the feedstock and cells was performed at, for example, between 200-750 RPM. In some examples, agitation was performed at 300 RPM. Agitation was provided by two or more impellers. Rushton, hydrofoil, and marine impellers may be used. Aeration was set between 0.1 and 5 vessel volumes per minute (VVM). In some examples, aeration was set at 0.5 VVM.

Dissolved oxygen (DO) was maintained, for example, between 30-90%. In some examples, DO was maintained at about 30%. The DO was maintained at this level by varying the agitation.

Fermentation was performed over the course of 5-21 days, for example. In some examples, fermentation was performed for 10 days.

Fermentation media was comprised of, for example, 50-100% DMR, 10-100% CDS, 50-100% corn mash, heavy oil production media or a combination thereof.

Oil Production media was used, for example. In some examples, NH₄NO₃ was added to the shake flasks at a similar concentration as Oil Production media. In some embodiments, maximum carbohydrate concentration was accompanied by glucoamylase concentrations of more than 175 g/L. In some embodiments, denaturation occurred.

In some examples, the feedstocks can be 50% corn mash and 50% Oil Production media. In some examples, the feedstocks can comprise no carbohydrate.

### Example 3: Carbohydrate and Oil Production Quantification

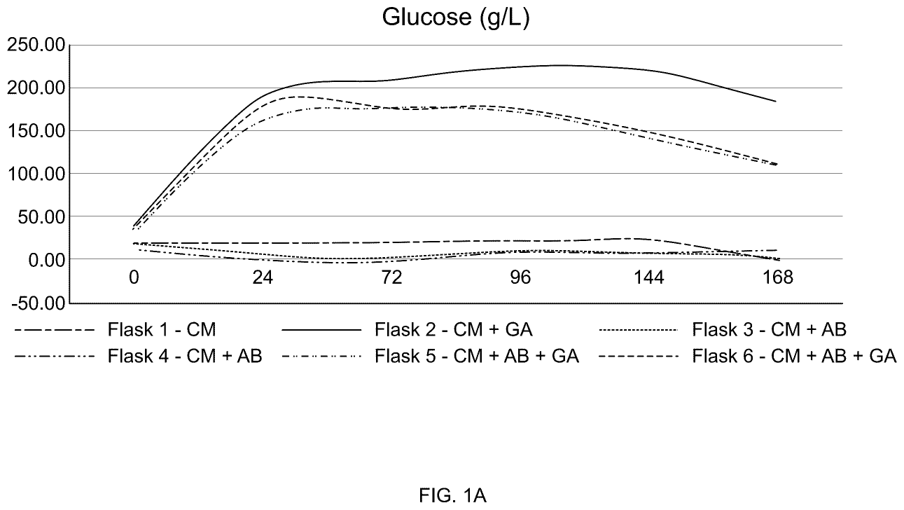

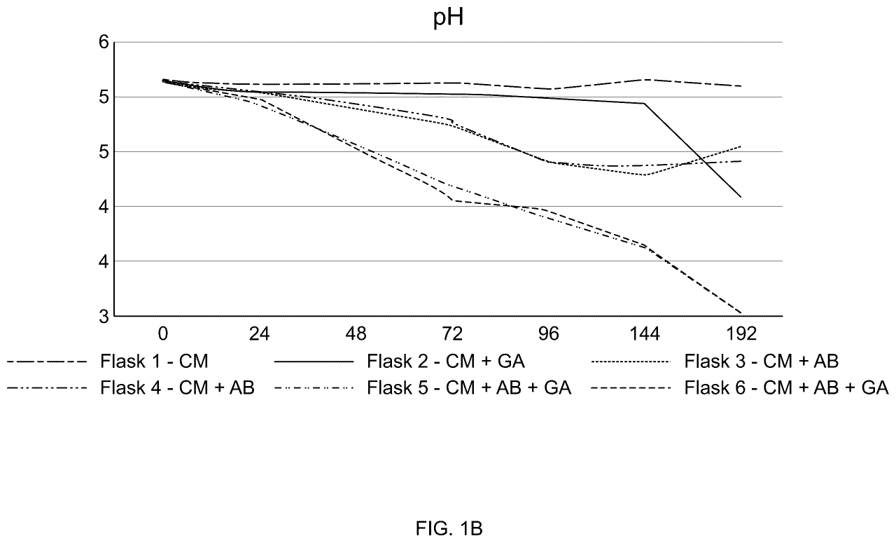

Carbohydrate concentrations in the fermentation broth were quantified using high-performance liquid chromatography (HPLC), for example as shown in FIG. 1A. As shown in FIG. 1A, the glucose concentration was measured for each flask between hours 0 and 168. For example, a Waters Arc system was equipped with an Aminex-HPX-87H column (Bio-Rad). The mobile phase was, for example, 5 mM sulfuric acid under isocratic conditions at 60° C.

In some examples, as shown in Table 2, CDS was used as a carbon nutritional source to test growth and production of oil. As shown in Table 2, various concentrations of Oil Production media, glucose, syrup, ammonia nitrate were used in media for culturing the A. pullulans cell population.

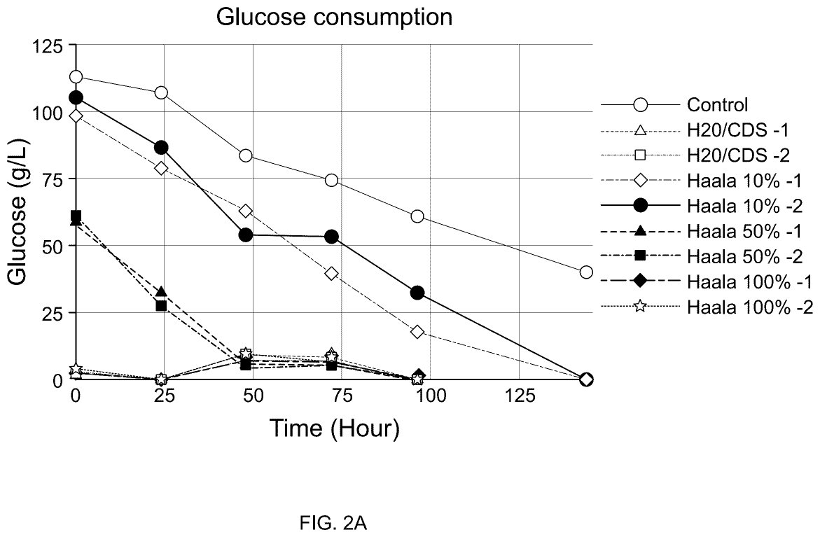

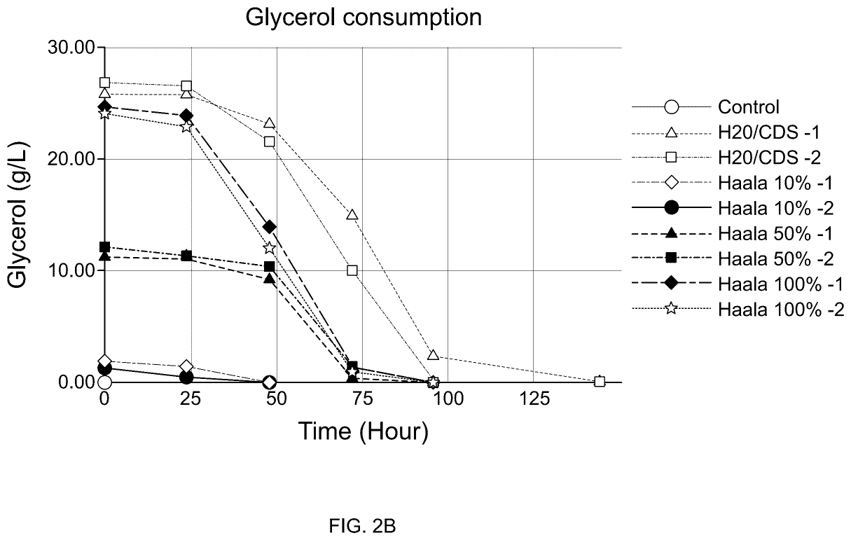

As illustrated in FIG. 2A, glucose consumption was measured for each media type, and for each flask along with the Oil Production media control over about 150 hours.

As illustrated in FIG. 2B, glycerol consumption was also measured for each media type, and for each flask along with a control over about 150 hours.

In some examples, Heavy oil was produced. In some examples, Heavy oil was produced with various carbon nutrition sources including glucose, sucrose, fructose, arabinose, xylose, mannitol, arabitol, HFCS, and evaporated beverage syrup. Oil in the fermentation broth was quantified either through gravity separation or solvent extraction, for example. For gravity separation, fermentation broth was allowed to settle overnight, and the heavy oil fraction was captured and weighed. For solvent extraction, fermentation broth was centrifuged, the supernatant was discarded, and the remaining oil and cell layer are mixed with a 1:1 ratio of solvent to broth. The sample was centrifuged, then the solvent and oil fraction were collected, and the solvent was evaporated, leaving pure oil.

In some examples, 50 mL shake flasks were used with a base Oil Production media, the base media having optimized conditions, for example with varying carbon nutrition sources such as carbohydrates at a concentration of 120 g/L and grown at a temperature of about 28° C.

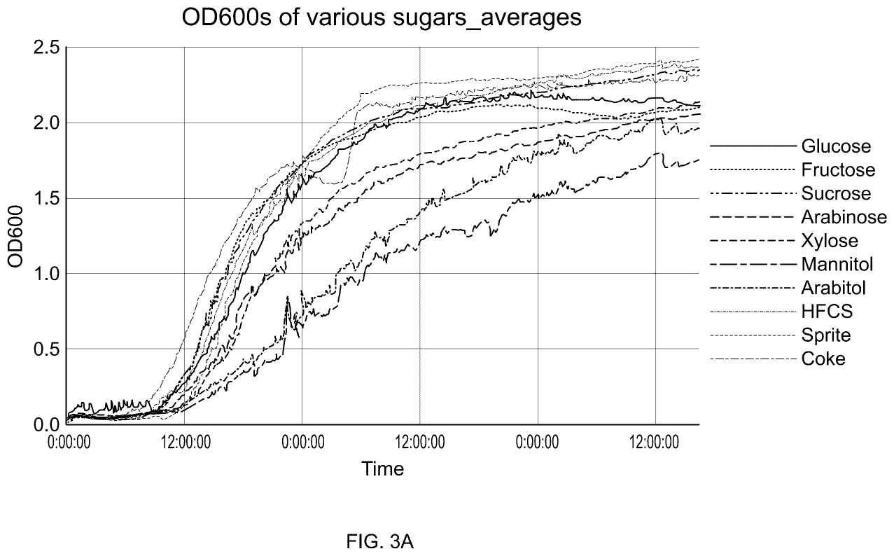

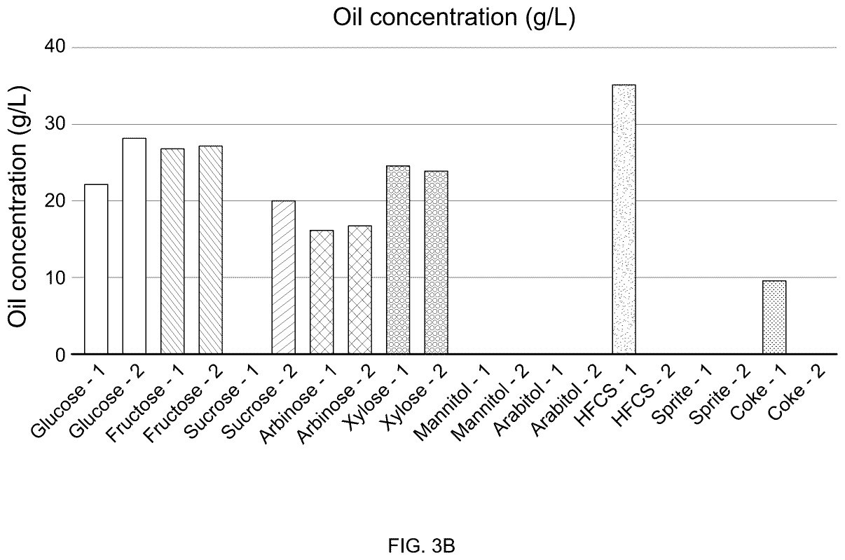

As illustrated in FIG. 3A, various populations of A. pullulans cultured in various carbon nutrition sources were measured for growth rate (OD₆₀₀).

### Example 4. Waste Sugar Utilization

In Example 4, batch fermentation experiments were performed using the melanin knockout strain A. pullulans (Δpks::ura3) grown on various waste sugar sources. A. pullulans was cultured in 50 mL shake flasks containing base Oil Production media with 120 g/L of each sugar source, including pure sugars such as glucose, fructose, sucrose, arabinose, and xylose, sugar alcohols including mannitol and arabitol, commercial syrups such as high fructose corn syrup (HFCS), and waste sugars from two different beverage waste sugar sources. Sugar consumption was quantified every 24 hours for 182 hours using HPLC analysis. Complete consumption was demonstrated across all tested sugar sources, confirming A. pullulans' ability to utilize diverse waste sugar feedstocks for heavy oil production.

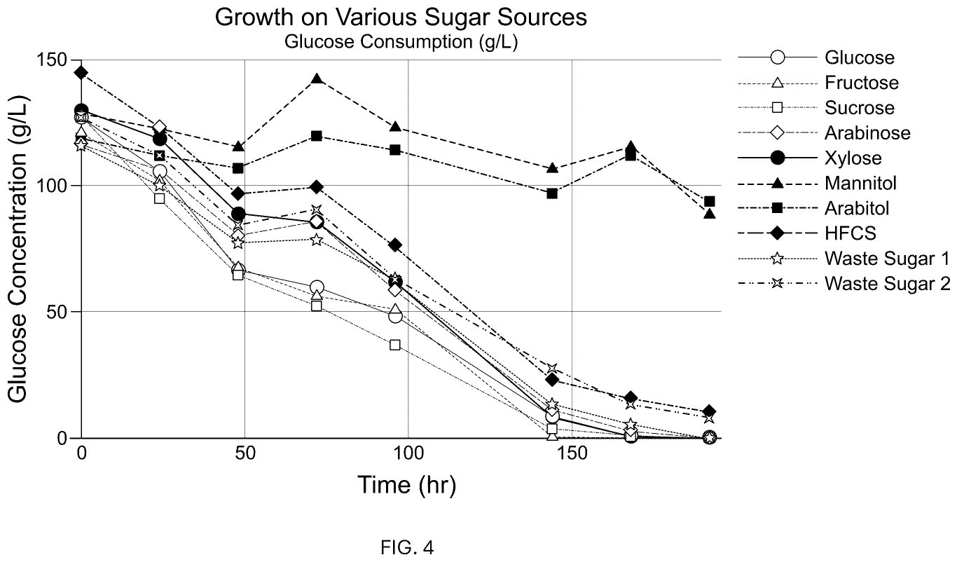

The results of Example 4 demonstrated that A. pullulans (Δpks::ura3) successfully consumed all tested sugar sources completely over the 182-hour experimental period. These results are shown in FIG. 4.

### Example 5. Crude Glycerol Co-Utilization

In Example 5, crude glycerol from renewable diesel production was characterized and tested as a feedstock using four different sugar combinations: 100% crude glycerol with 0% glucose, 50% crude glycerol with 50% glucose, 10% crude glycerol with 90% glucose, and 0% crude glycerol with 100% glucose as a control. A. pullulans (Δpks::ura3) was grown in 50 mL shake flasks in duplicate for each condition, with both glucose and glycerol concentrations quantified every 24 hours for 192 hours. Complete consumption of both glucose and glycerol was achieved across all conditions, with the highest heavy oil production observed at intermediate glycerol concentrations, demonstrating the effectiveness of crude glycerol as a co-substrate for heavy oil production.

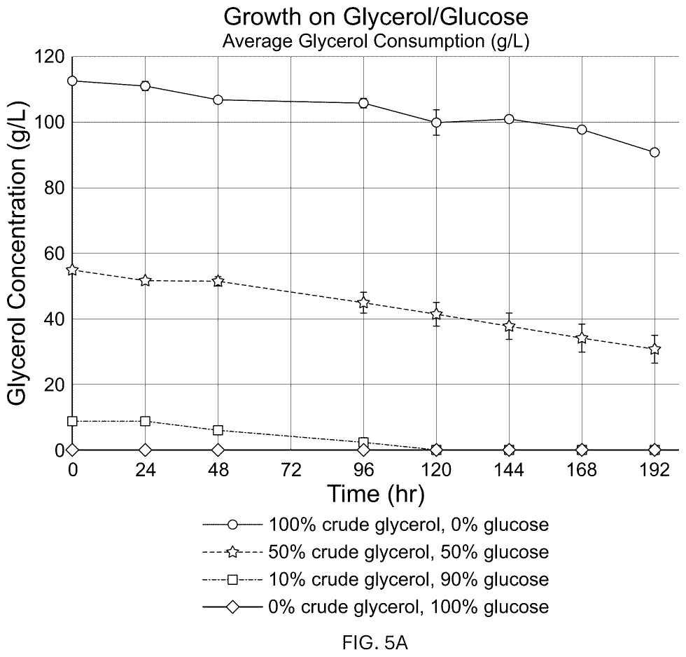

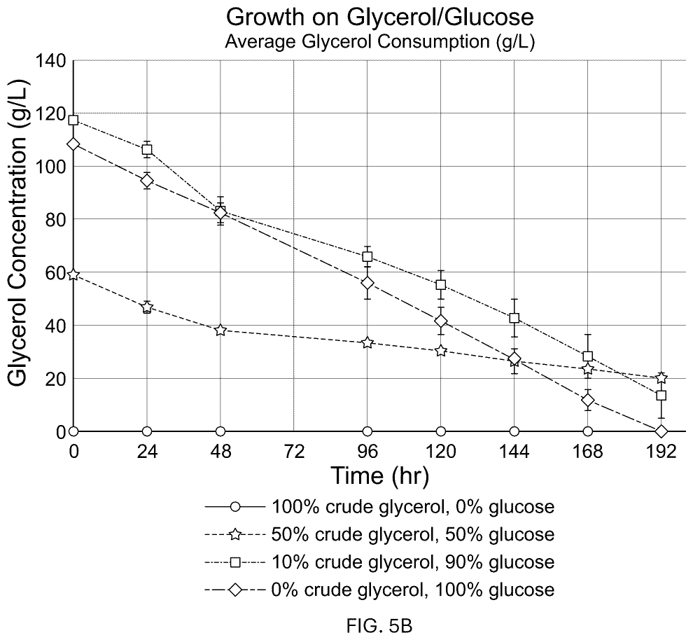

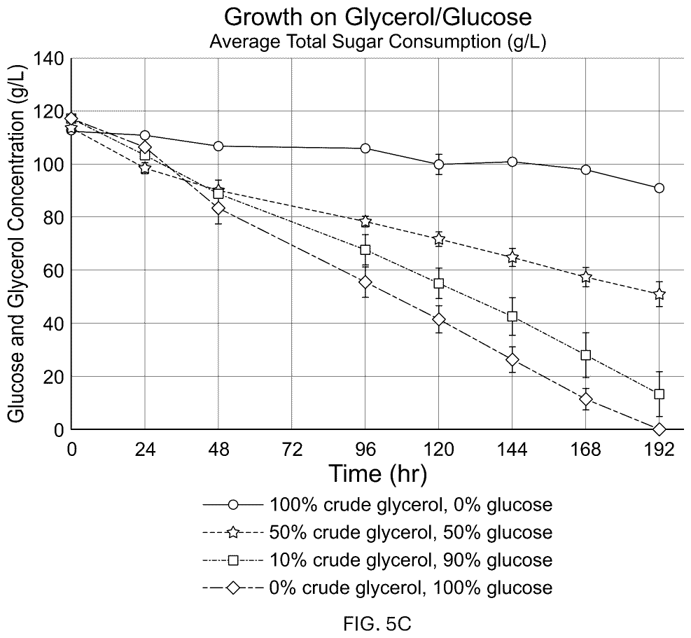

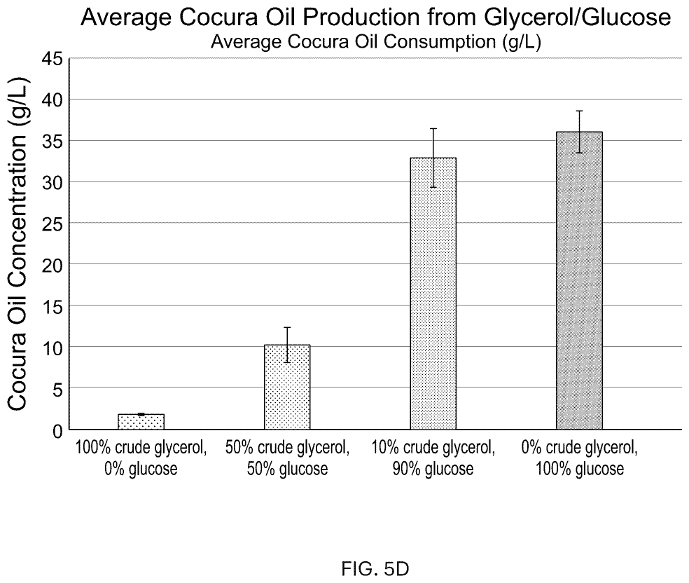

The results are shown in FIGS. 5A to 5D. Complete consumption of both glucose and glycerol was achieved across all tested ratios, demonstrating A. pullulans' capability to effectively co-utilize these carbon sources. Heavy oil production data revealed good performance at intermediate glycerol concentrations, indicating synergistic effects between glucose and glycerol metabolism.

### Example 6. Corn Mash Processing and Utilization

In Example 6, corn mash was pre-treated with glucoamylase overnight to release glucose, then processed under four different conditions: all solids retained, some solids removed, no solids (centrifuged), and sugar only (highly filtered). Each condition was normalized to 120 g/L glucose and used in base Oil Production media without added nitrogen source due to corn mash's high nitrogen content 1. A. pullulans (Δpks::ura3) was cultured in 50 mL shake flasks in triplicate, with glucose consumption monitored every 24 hours for 210 hours. Oil production was quantified for each processing condition, demonstrating successful utilization of corn mash across all processing variants and confirming the versatility of corn mash as a feedstock regardless of solids content.

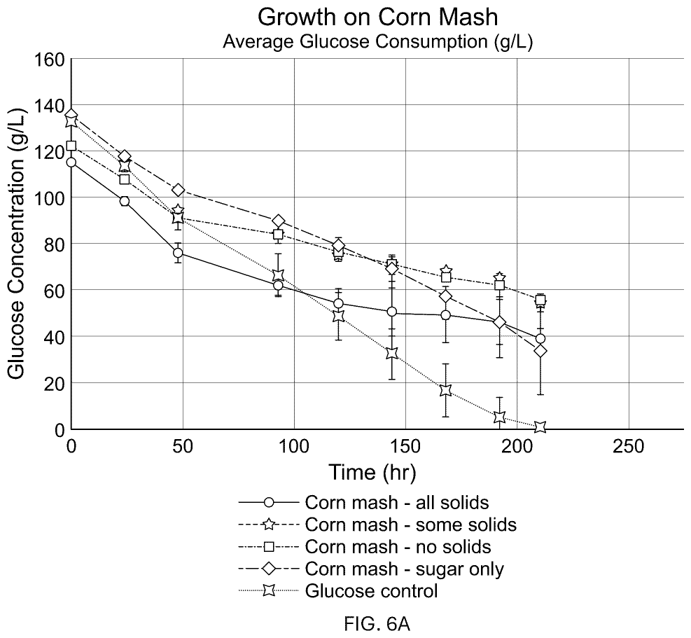

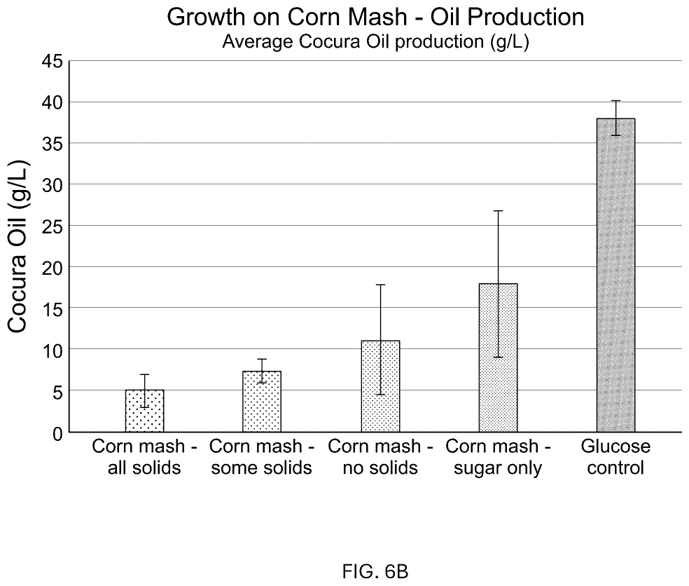

The results of Example 6 are shown in FIGS. 6A and 6B. In Example 6, glucose consumption was successfully achieved across all corn mash processing conditions over the 210-hour experimental period, with complete substrate utilization demonstrated regardless of solids content. Heavy oil production was observed in all conditions, indicating that corn mash can serve as an effective feedstock for heavy oil production without requiring extensive solids removal or purification steps.

### Example 7. Condensed Distillers Solubles (CDS) Utilization

In Example 7, five experimental conditions were evaluated using CDS from corn ethanol production: 100% Oil Production media as control, 50% CDS with 50% Water, 10% CDS with 90% Oil Production media, 50% CDS with 50% Oil Production media, and 100% CDS with 0% Oil Production media. A. pullulans (Δpks::ura3) was grown in 50 mL shake flasks with glucose and glycerol concentrations tracked using HPLC 1. Complete consumption of both glucose and glycerol was achieved in all conditions, demonstrating successful growth on CDS as a standalone feedstock and confirming its potential as a zero carbon intensity score feedstock for sustainable aviation fuel production.

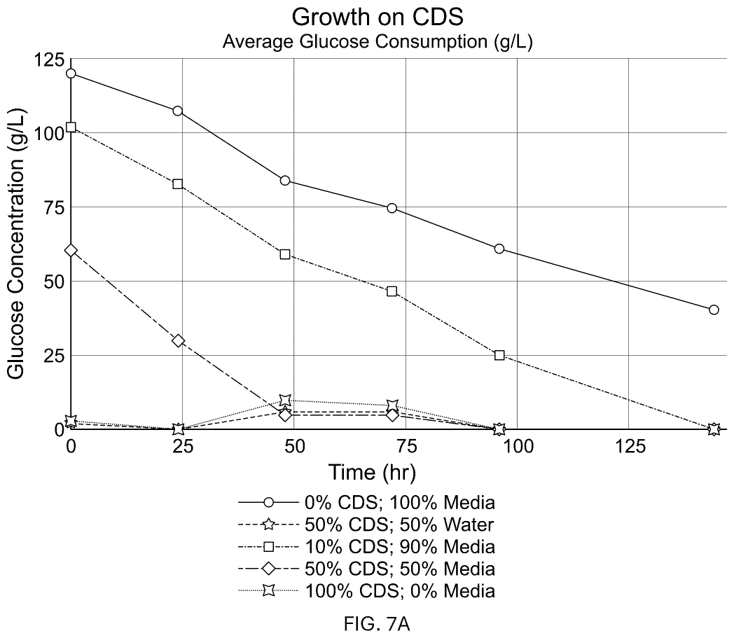

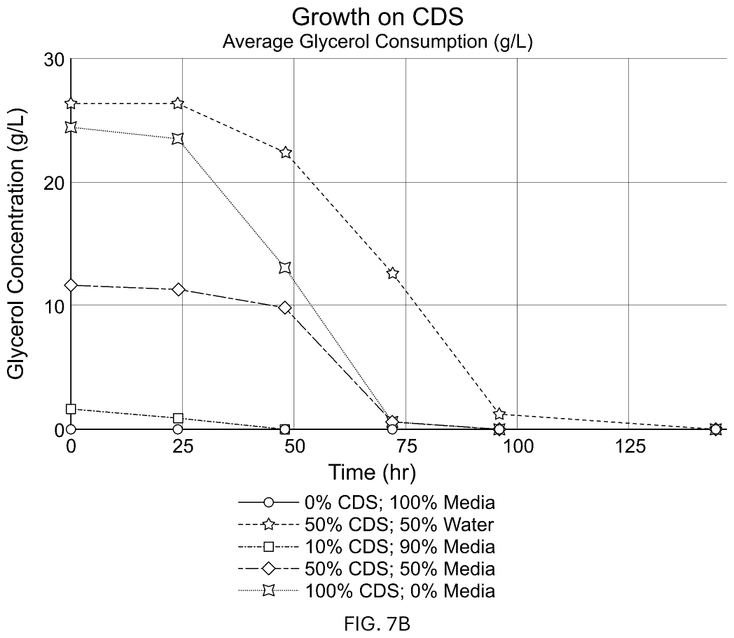

The results are shown in FIGS. 7A and 7B. In Example 7, consumption of both glucose and glycerol was achieved across various CDS concentration conditions, including 100% CDS as the sole nutrient source, demonstrating the suitability of CDS as a standalone feedstock. The successful growth and substrate utilization across varying CDS concentrations validates its nutritional adequacy for A. pullulans cultivation.

### Example 8. Lignocellulosic Hydrolysate Processing and Adaptive Laboratory Evolution

In Example 8, deacetylated and Mechanically Refined (DMR) corn stover hydrolysate was tested at two concentrations with distinct performance outcomes. At 50% DMR concentration, complete co-utilization of glucose, xylose, and arabinose was achieved over 7 days, consuming 76 g/L total sugar at a rate of 0.46 g/L/h, while at 75% DMR concentration, consumption of 115.2 g/L hydrolysate sugars occurred over 15 days at a reduced rate of 0.34 g/L/h due to inhibitor effects. To overcome inhibitor challenges, A. pullulans (Δpks::ura3) was subjected to adaptive laboratory evolution (ALE) by passaging for 20-30 days in 75% DMR medium, selecting faster-growing isolates with each passage. Twenty ALE isolates were generated and tested using plate reader analysis over 96 hours, with all 20 isolates demonstrating enhanced tolerance compared to the parent strain, achieving higher optical density in DMR hydrolysate conditions and confirming the success of the ALE approach for improving strain performance on challenging lignocellulosic feedstocks.

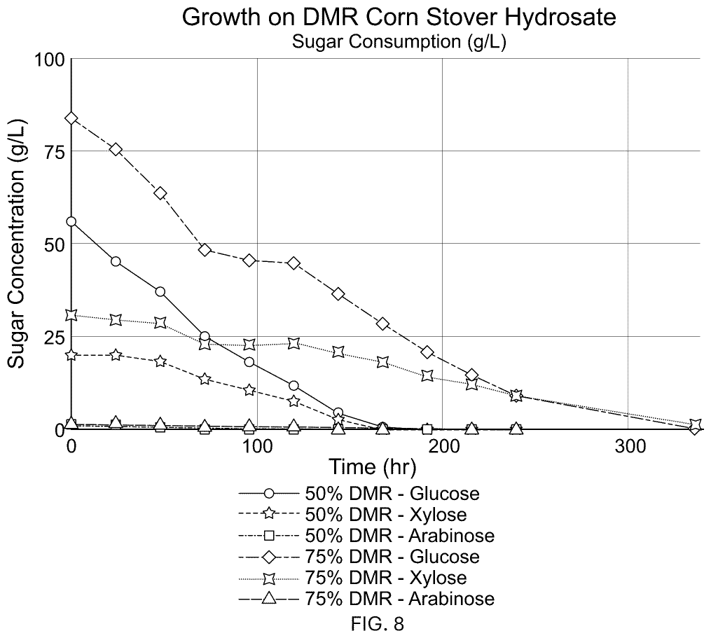

The results are shown in FIG. 8. The experiments in Example 8 demonstrated successful co-utilization of multiple lignocellulosic sugars (glucose, xylose, and arabinose) by A. pullulans, with consumption rates inversely related to hydrolysate concentration due to inhibitor effects 1. The ALE program proved highly successful, with all 20 generated isolates showing improved tolerance to DMR hydrolysate inhibitors compared to the parent strain. This strain improvement approach effectively addressed the primary limitation of lignocellulosic feedstock utilization (e.g., inhibitor toxicity) while maintaining the organism's native ability to co-metabolize multiple sugar types.

### Example 9. Analytical Methods and Oil Quantification

In Example 9, sugar quantification was performed using a Waters Arc system equipped with an Aminex-HPX-87H column (Bio-Rad) with 5 mM sulfuric acid mobile phase under isocratic conditions at 60° C. Oil quantification was accomplished through two distinct methods: gravity separation, where fermentation broth was allowed to settle overnight and the heavy oil fraction was captured and weighed, and solvent extraction, where fermentation broth was centrifuged and the oil and cell layer were mixed with a 1:1 ratio of solvent to broth, followed by collection of the solvent and oil fraction and solvent evaporation to yield pure oil 1. These analytical protocols provided reliable and reproducible methods for quantifying both substrate consumption and product formation across all feedstock types, enabling accurate assessment of fermentation performance and oil production yields throughout the experimental program.

The analytical methods of Example 9 provided accurate and reproducible quantification of both substrate consumption and product formation across all tested feedstock types. The HPLC system effectively resolved and quantified multiple sugar components simultaneously, including glucose, xylose, arabinose, and glycerol, enabling comprehensive monitoring of co-substrate utilization patterns. Both gravity separation and solvent extraction methods proved effective for oil quantification, with gravity separation offering simplicity for routine monitoring and solvent extraction providing precision for detailed product characterization.

### ADDITIONAL EMBODIMENTS

The following exemplary embodiments are provided, the order of which is not to be construed as designating levels of importance:

In some embodiments, disclosed herein is a method of producing sustainable heavy oils comprising: producing one or more feedstocks comprising deacetylated and mechanically refined (DMR) grain-based hydrolysate media; adding one or more nutritional carbon sources to the DMR media feedstocks, wherein the one or more nutritional sources comprises one or more carbohydrates; and facilitating growth of a cell population comprising Aureobasidium pullulans (A. pullulans) or a genetically modified version thereof.

In some embodiments, the DMR lignocellulosic hydrolysate media comprise one or more of hydrolysate media, corn-derived media, carbohydrate-derived media, or any combination thereof. In some embodiments, the sugar-derived media comprises sugar beet-derived media, sugarcane-derived media or corn-derived media, or any combination thereof. In some embodiments, the DMR lignocellulosic hydrolysate media comprise manufacturing byproducts. In some embodiments, the manufacturing byproducts comprises waste byproducts.

In some embodiments, the manufacturing byproducts comprises byproducts of fuel production, food production, beverage production, livestock feed production, cosmetics production, or any combination thereof. In some embodiments, the fuel production comprises ethanol production. In some embodiments, the beverage production comprises soft drink production. In some embodiments, the corn-derived media comprises corn mash media. In some embodiments, the corn-derived media comprises condensed distillers solubles (CDS) media. In some embodiments, the carbohydrate-derived media comprises sugar syrup media. In some embodiments, the sugar syrup media comprises beverage-derived syrup. In some embodiments, the beverage-derived syrup comprises byproduct syrup. In some embodiments, the one or more carbohydrates comprises one or more of: glucose, sucrose, fructose, arabinose, xylose, high fructose corn syrup, or any combination thereof.

In some embodiments, the method further comprises extracting the produced heavy oils. In some embodiments, the method further comprises culturing the A. pullulans cell population under selective pressure conditions.

In some embodiments, the selective pressure conditions comprises selection for faster-growing isolates of the A. pullulans cell population.

In some embodiments, the method further comprises evaluating the fitness of the A. pullulans cell population. In some embodiments, the one or more carbohydrates comprises between about 10% and 99% of the one or more feedstocks. In some embodiments, the DMR lignocellulosic hydrolysate media comprise oil production media.

In some embodiments, the method further comprises modifying the pH of the one or more feedstocks after the growth of the A. pullulans cell population. In some embodiments, modifying the pH comprises raising pH levels.

In some embodiments, the feedstocks comprise low carbon intensity (CI) feedstocks. In some embodiments, the heavy oil comprises low carbon intensity (CI) heavy oil.

In some embodiments, the method produces a higher yield of the heavy oil compared to methods not culturing the A. pullulans or a genetically modified derivative thereof in the feedstock.

In some embodiments, the A. pullulans cell population, or a genetically modified derivative thereof, produce heavy oil more efficiently compared to methods not culturing the A. pullulans or a genetically modified derivative thereof in the feedstock.

In some embodiments, the A. pullulans cell population, or a genetically modified derivative thereof, comprise increased environmental tolerance characteristics as compared to an A. pullulans cell population, or a genetically modified derivative thereof, not cultured in the feedstock.

In some embodiments, the increased environmental tolerance characteristics comprise tolerance to environmental toxins, lack of environmental nutrition, or both.

Disclosed herein in another embodiment is a feedstock for use in heavy oil production, the feedstock comprising: one or more deacetylated and mechanically refined (DMR) lignocellulosic hydrolysate media; and one or more nutritional carbon sources, wherein the one or more nutritional sources comprises one or more carbohydrates.

In some embodiments, the DMR lignocellulosic hydrolysate media comprises one or more of hydrolysate media, corn-derived media, carbohydrate-derived media, or any combination thereof. In some embodiments, the one or more carbohydrates comprises carbohydrate syrup. In some embodiments, the carbohydrate syrup comprises one or more of sugar beet-derived syrup, sugarcane-derived syrup, or corn-derived syrup, or any combination thereof. In some embodiments, the carbohydrate syrup comprises glucose, sucrose, fructose, arabinose, xylose, high fructose corn syrup, or any combination thereof. In some embodiments, the carbohydrate syrup comprises a first-generation carbohydrate syrup.

In some embodiments, the first-generation carbohydrate syrup is derived at least in part from corn grain or ethanol-producing plants. In some embodiments, the carbohydrate syrup comprises a second-generation carbohydrate syrup. In some embodiments, the second-generation carbohydrate syrup comprises a waste carbohydrate syrup. In some embodiments, the waste carbohydrate syrup is derived at least in part from agricultural waste, food waste, beverage waste, energy production crop waste, or any combination thereof. In some embodiments, the waste carbohydrate syrup comprises an off-specification carbohydrate syrup. In some embodiments, the beverage waste comprises off-specification beverage syrup.

In some embodiments, the one or more carbohydrates comprise between about 10% and 99% of the one or more feedstocks.

In some embodiments, the feedstocks comprise low carbon intensity (CI) feedstocks. In some embodiments, the heavy oil comprises low carbon intensity (CI) heavy oil.

In some embodiments, the feedstock is used to support culturing A. pullulans or a genetically modified derivative thereof to produce a higher yield of the heavy oil compared to non-feedstock media.

In some embodiments, the feedstock is used to support culturing A. pullulans cell population, or a genetically modified derivative thereof, to produce heavy oil more efficiently compared to non-feedstock media.

In some embodiments, the feedstock is used to support culturing A. pullulans cell population, or a genetically modified derivative thereof, to comprise increased environmental tolerance characteristics as compared to an A. pullulans cell population, or a genetically modified derivative thereof, not cultured in the feedstock.

In some embodiments, the increased environmental tolerance characteristics comprise tolerance to environmental toxins, lack of environmental nutrition, or both.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

What is claimed is:

1. A method of producing heavy oil comprising:
providing one or more feedstocks comprising deacetylated and mechanically refined (DMR) lignocellulosic hydrolysate media and one or more nutritional carbon sources, wherein the one or more nutritional sources comprises one or more carbohydrates;
culturing a cell population comprising Aureobasidium pullulans (A. pullulans), or a genetically modified version thereof, on the one or more feedstocks; and
generating the heavy oil using the cell population.

2. The method of claim 1, wherein the DMR lignocellulosic hydrolysate media comprise one or more of hydrolysate media, corn-derived media, carbohydrate-derived media, or any combination thereof.

3. The method of claim 2, wherein the DMR lignocellulosic hydrolysate media comprise manufacturing byproducts selected from the group consisting of byproducts of fuel production, food production, beverage production, livestock feed production, cosmetics production, or any combination thereof.

4. The method of claim 2, wherein the corn-derived media comprises corn mash media or CDS media.

5. The method of claim 1, further comprising extracting the produced heavy oil.

6. The method of claim 1, further comprising culturing the A. pullulans cell population or the genetically modified version thereof under selective pressure conditions, wherein the selective pressure conditions comprise selection for faster-growing isolates of the A. pullulans cell population.

7. The method of claim 1, further comprising evaluating the fitness of the A. pullulans cell population.

8. The method of claim 1, wherein the one or more carbohydrates comprise between about 10% and 99% of the one or more feedstocks.

9. The method of claim 1, wherein the feedstocks comprise low carbon intensity (CI) feedstocks.

10. The method of claim 1, wherein the heavy oil comprises low carbon intensity (CI) heavy oil.

11. The method of claim 1, wherein the method produces a higher yield of the heavy oil compared to methods not culturing the A. pullulans or a genetically modified derivative thereof in the feedstock.

12. The method of claim 1, wherein the A. pullulans cell population, or a genetically modified derivative thereof, produce heavy oil more efficiently compared to methods not culturing the A. pullulans or a genetically modified derivative thereof in the feedstock.

13. The method of claim 1, wherein the A. pullulans cell population, or a genetically modified derivative thereof, comprise increased environmental tolerance characteristics as compared to an A. pullulans cell population, or a genetically modified derivative thereof, not cultured in the feedstock, wherein the increased environmental tolerance characteristics comprise tolerance to environmental toxins, lack of environmental nutrition, or both.

14. A feedstock for use in heavy oil production, the feedstock comprising:
one or more deacetylated and mechanically refined (DMR) lignocellulosic hydrolysate media; and
one or more nutritional carbon sources, wherein the one or more nutritional sources comprise one or more carbohydrates.

15. The feedstock of claim 14, wherein the DMR lignocellulosic hydrolysate media comprise one or more of hydrolysate media, corn-derived media, carbohydrate-derived media, or any combination thereof.

16. The feedstock of claim 14, wherein the one or more carbohydrates comprise a carbohydrate syrup comprises a first-generation carbohydrate syrup derived at least in part from corn grain or ethanol-producing plants.

17. The feedstock of claim 14, wherein the one or more carbohydrates comprise a carbohydrate syrup comprises a second-generation sugar syrup, and wherein the second-generation carbohydrate syrup comprises a waste carbohydrate syrup derived at least in part from agricultural waste, food waste, beverage waste, energy production crop waste, or any combination thereof.

18. The feedstock of claim 14, wherein the feedstock is used to support culturing A. pullulans or a genetically modified derivative thereof to produce a higher yield of the heavy oil compared to non-feedstock media.

19. The feedstock of claim 14, wherein the feedstock is used to support culturing A. pullulans cell population, or a genetically modified derivative thereof, to produce heavy oil more efficiently compared to non-feedstock media.

20. The feedstock of claim 14, wherein the feedstock is used to support culturing A. pullulans cell population, or a genetically modified derivative thereof, to comprise increased environmental tolerance characteristics as compared to an A. pullulans cell population, or a genetically modified derivative thereof, not cultured in the feedstock, wherein the increased environmental tolerance characteristics comprise tolerance to environmental toxins, lack of environmental nutrition, or both.
